(19) United States
(12) Patent Application Publication
(10) Pub. No.: US 2003/0170275 A1
(43) Pub. Date: Sep. 11, 2003

(54) Treatment of conditions of the central nervous system using mycobacteria

(76) Inventors: Graham A.W.I. Rook, London (GB); Christopher Alan Lowry, Avon (GB); Stafford Louis Lightman, Avon (GB)

Correspondence Address: Nixon & Vanderhye, 8th Floor, 1100 North Glebe Road, Arlington, VA, 22201-4714, US

(21) Appl. No.: 10/258,550

(22) PCT Filed: Apr. 27, 2001
(86) PCT No.: PCT/GB01/01867

(30) Foreign Application Priority Data
Apr. 28, 2000 (GB) 0010496.8

(51) Int. Cl. A61K 39/04

(52) U.S. Cl. 424/248.1

(57) ABSTRACT

Treatment of conditions of the central nervous system, such as pain, mental disorders such as schizophrenia and stress-related disorders such as depression, using mycobacterial material, especially M. vaccae material.

This invention relates to the treatment of conditions of the central nervous system using compositions based on mycobacteria such as M. vaccae.

Mycobacteria, particularly M. vaccae, have been employed in the treatment of various disorders.

GB 2156673 (W085/03639) describes immunotherapeutic agents comprising killed cells of M. vaccae. These agents are useful in the immunotherapy of mycobacterial disease, especially tuberculosis and leprosy. The use of this immunotherapeutic agent is said to facilitate the removal of the persisting bacilli responsible for tuberculosis or leprosy which, as is well known, it is difficult to remove by chemotherapy alone.

W085/05034 describes compositions for the alleviation of the symptoms of, and for the treatment or diagnosis of, arthritic disease which comprise as active ingredient the whole organism of M. vaccae. Preparations of M. vaccae are said to be useful for the treatment of various auto-immune diseases and especially arthritic conditions including rheumatoid arthritis, ankylosing spondylitis or Reiter's syndrome.

W091/02542 refers to compositions comprising antigenic and immunoregulatory material derived from M. vaccae as being generally useful in the treatment of pathological conditions in which the proportion of agalactosyl IgG (i.e. IgG which lacks terminal galactose from the N-linked oligosaccharides on the heavy chains) is increased.

W092/08484 refers to the use of M. vaccae for the treatment of uveitis.

W094/06466 provides the use of antigenic and/or immunoregulatory materials derived from M. vaccae for the manufacture of a medicament useful in the therapy of AIDS and also in the therapy of HIV-positive asymptomatic patients.

PCT/GB95/00715 teaches that immunotherapy with M. vaccae is expected to be effective against tumours of mesodermal, endodermal and ectodermal origin, including breast and bronchial tumours.

PCT/GB98/03346 teaches cold shock treatment of M. vaccae to induce a cold shock response which results in the production of cold shock proteins. Preparations of cold shocked M. vaccae are useful in the treatment of autoimmune disease, chronic inflammatory disease and conditions associated with exposure to cold including Raynaud's phenomenon, Raynaud's disease, hypothermia and frostbite.

PCT/GB00/0054 teaches the use of M. vaccae in the treatment of viral infections, particularly chronic viral infections.

WO93/16727 teaches that Schizophrenia may be associated with an auto-immune reaction caused by a past or cryptic infection and suggests that M. vaccae may be useful in the treatment of schizophrenia and other mental diseases where these are associated with such an auto-immune reaction resulting from a cryptic infection.

PCT/GB97/03460 teaches the use of M. vaccae to treat Chronic Fatigue Syndrome associated with a shift in activity of the immune system from Th1 activity to Th2 activity.

The present inventors have discovered that administering M. vaccae activates various parts of the brain in a highly selective manner, leading to the modulation of functions of the central nervous system (CNS).

Conventional approaches to therapeutic modulation of CNS function involve the use of drugs that target enzymes or receptors within the brain. The success of this approach is limited by the multiple, often conflicting roles of each mediator in different brain areas. For example, serotonergic neurones in different areas can be involved in down-regulation or up-regulation of pain perception. The existence of different receptors for the same mediator in different brain areas provides some additional targets, but specificity of targeting remains a problem, as does access of the compound to the brain.

Peripheral inflammation, either local inflammation in viscera or inflammatory signals (cytokines) in the blood, can activate stress response pathways in the brain. This is thought to be mediated in the first case by sensory afferent neurones within the vagus nerve, and in the second case by communication across the blood-brain barrier at specialised sites, for example at the area postrema (AP). These signals converge to activate the body's defence mechanisms including an activation of the ^{˜}stress^{˜} neuroendocrine axis, most commonly measured as an increase in the glucocorticoid hormone corticosterone (cortisol in primates and humans). It is believed that the nucleus of the solitary tract (nTS) serves as a ^{˜}common gateway^{˜} through which inflammatory signals act to alter brain and endocrine function.

Signals from the nTS are then secondarily relayed to the rest of the brain by neurones which are known to project both directly and indirectly to the brain region in the hypothalamus (the paraventricular nucleus of the hypothalamus; PVN) that regulates the stress neuroendocrine axis (hypothalamo-pituitary adrenal (HPA) axis).

The present inventors have shown that this stress response pathway is activated by administering Mycobacterial preparations.

Surprisingly, the present inventors have also discovered that preparations of Mycobacteria such as M. vaccae can activate specific areas of the brain using previously unknown neural pathways. These novel pathways relay signals of inflammation from the periphery to highly selective areas of the brain.

The areas activated by M. vaccae are involved in much human psychopathology. The present invention thus provides preparations of Mycobacteria, such as M. vaccae, for treating a range of conditions and mental disorders associated with these activated areas of the brain, methods of treatment of these conditions involving Mycobacteria and the use of mycobacteria in such methods of treatment.

One aspect of the present invention is the use of a Mycobacterium in the manufacture of a medicament for use in a method of treating a condition of the central nervous system (CNS).

Conditions of the CNS may include pain, mental disorders such as schizophrenia, and stress-related disorders such as depression. In preferred embodiments of the present invention, the method may comprise stimulating a peripheral sensory afferent nerve by administration of Mycobacteria such as M. vaccae.

The present invention also provides a method of selectively activating defined areas of the brain and spinal cord associated with conditions of the central nervous system, comprising the step of stimulating a peripheral sensory afferent nerve.

Afferent nerves stimulated by methods according to the present invention may be associated with the vagus nerve, the dorsal root ganglia or other neural pathway.

Areas of the brain and spinal cord associated with conditions of the CNS which are activated by such stimulation are described herein.

Areas of the brain and spinal cord associated with conditions of the CNS do not include the nucleus Tractus Solitarius (nTS), which has been implicated in the relay of inflammation signals to the hypothalamo-pituitary-adrenal axis.

Neurones within the ^{˜}descending analgesia^{˜} system and the ^{˜}diffuse noxious inhibitory contols^{˜} (DNIC) system are activated by M. vaccae. These are known pain inhibitory systems.

The present invention thus provides the use of a Mycobacterium preparation in the manufacture of a medicament for the treatment of pain.

Compositions of the present invention may be particularly useful in the treatment of lower back pain and pleural pain in patients with Mesothelioma.

Noradrenergic and serotonergic systems associated with stress and major depression are also activated by M. vaccae. The dorsomedial hypothalamic nucleus plays a role in depression, anxiety and regulation of feeding. The activation of this area by M. vaccae is therefore significant for the treatment of a range of stress related disorders.

The present invention thus provides the use of a Mycobacterium preparation in the manufacture of a medicament for use in the treatment of stress-related psychiatric disorders.

Stress-related psychiatric disorders may include depression, anxiety, panic disorder, and eating disorders such as anorexia nervosa and bulimia.

The dorsomedial hypothalamic nucleus is strongly implicated in Schizophrenia. By enhancing dopamine or serotonin synthesis within this region, M. vaccae may be useful in the treatment of this disorder.

The present invention thus provides the use of a Mycobacterium preparation in the manufacture of a medicament for use in a method of treating schizophrenia.

The present invention also provides a method of treatment of pain, stress related disorders or schizophrenia comprising administering to a patient an effective amount of a therapeutic composition comprising material derived from a Mycobacterium.

Mycobacteria suitable for use preparations for treating schizophrenia may be ultrasonically disrupted and/or adsorbed onto an inert support, for example, nitrocellulose.

The treatment of schizophrenia which is not associated with an auto-immune reaction is also encompassed by the present invention.

Schizophrenia may be treated according to the present invention by stimulating peripheral sensory afferent nerves.

Peripheral afferent nerves may be stimulated according to some embodiments of this aspect of the present invention by administration of mycobacteria to the respiratory tract. Mycobacteria may be administered to the respiratory tract by conventional methods which may include intra-tracheal injection.

A peripheral sensory afferent nerve stimulated in accordance with the present invention may have a terminal located in the skin, respiratory tract or gastrointestinal tract.

Material for use in mycobacterial preparations for the stimulation of sensory afferent nerves in accordance with the present invention may include whole, killed mycobacterial cells, treated mycobacterial cells, mycobacterial cell extracts, fractions or components or pharmacologically active substances which are not derived from mycobacteria.

Material suitable for use in mycobacterial preparations may include mycobacterial cells adsorbed onto a support, for example nitrocellulose particles, before administration. Cells may be treated before being used for stimulation, such treatment may include ultrasonic disruption.

Material including fractions, extracts or components of mycobacterial cells may also be used to stimulate neural afferent terminals. Such material may be prepared and/or purified according to standard techniques. Suitable mycobacterial material may have an indirect effect either as an antigen or pharmacological agent that stimulates release of a mediator which affect afferent nerve terminals, or a direct effect as a pharmacological agent acting directly on the nerve terminals.

A mycobacterium suitable for use in preparations, compositions and methods of the present invention is M. vaccae.

A Mycobacterium vaccae preparation for use stimulating neural afferent terminals may, for example, include material which can be or include dead cells of M. vaccae. Such cells may be killed, for instance using irradiation, e.g. from ⁶⁰Cobalt at a dose of 2.5 megarads, chemically, or by any other means, although autoclaving is preferred, e.g. at 69 kPa for 10 minutes at 115° C.-125° C. Autoclaving may yield a more effective preparation than irradiation.

M. vaccae cells may be disrupted prior to administration, for example by ultrasonication.

A suitable material for use in an M. vaccae preparation may comprise whole dead cells or a fraction thereof as described above. Instead of killed cells, other material derived from M. vaccae may be used, in particular an extract or a synthetic molecule which has the requisite activity.

SRL172 is a M. vaccae formulation derived from the strain denoted R877R which was deposited under the Budapest Convention at the National Collection of Type Cultures (NCTC) Central Public Health Laboratory, Colindale Avenue, London NW9 5HT, United Kingdom, on Feb. 13, 1984 under the number NCTC 11659. R877R was originally isolated from mud samples from the Lango district of Central Uganda (Stanford and Paul).

SRL172 is an example of a formulation suitable for use in accordance with the present invention. Other suitable formulations for use in accordance with the present invention may be derived from species and strains of Mycobacteria other than M. vaccae NCTC 11659. An organism can be identified as belonging to Mycobacteria, or more precisely to a species such as M. vaccae by biochemical and antigenic criteria (Bonicke et al.), or by molecular methods such as PCR and restriction enzyme analysis as described by Telenti et al.

Prior to being killed and/or disrupted, M. vaccae cells may be grown on a suitable solid medium. A modified Sauton=s liquid medium may be preferred (Boyden et al.), solidified with agar, preferably 1.3% agar. After aerobic incubation, generally at 32° C. for 10 days, the organisms may be harvested, then weighed and suspended in diluent, ready for administration. Storage, if required before use, may be at 4° C.

Instead of growing the cells on a solid medium, a liquid medium, such as the modified Sauton's medium (Boyden et al.), may be employed, for instance in a fermentor.

The diluent may be unbuffered saline, pyrogen-free.

Preferably, the diluent is borate-buffered, preferably containing a surfactant such as Tween 80⁷. A suitable borate buffer is: Na₂B₄O₇.10H₂O—3.63 g, H₃BO₃—5.25 g, NaCl—6.19 g, Tween 80⁷ 0.0005%, distilled water to 1 litre. These diluents are pharmaceutically acceptable.

Mycobacterial preparations may also be used in the formulation of pharmaceutical compositions and medicaments for use in accordance with the present invention.

It is preferred for the present invention that a mycobacterial preparation is administered free or substantially free from non-mycobacterial antigenic or immunoregulatory material. In other words the medicament or composition to be administered may include, or may consist essentially of, mycobacterial preparation, such as dead cells, an extract or derivative thereof, and a pharmaceutically acceptable diluent. A preferred mycobacterium for use in such preparations is M. vaccae.

Administration is preferably in a “therapeutically effective amount”, this being sufficient to show benefit to a patient. Such benefit may be at least amelioration of at least one symptom. The actual amount administered, and rate and time-course of administration, will depend on the nature and severity of what is being treated. Prescription of treatment, e.g. decisions on dosage etc, is within the responsibility of general practitioners and other medical doctors.

A single dosage (where dead cells are to be administered) will generally contain from 10⁷ to 10¹⁰ killed mycobacteria microorganisms. Patients may be administered a single dose of 10⁷ to 10⁹ disrupted mycobacteria, though the dose may be repeated if need be, for instance at intervals from 2 weeks to 6 months.

A composition may be administered alone or in combination with other treatments, either simultaneously or sequentially dependent upon the condition to be treated.

Pharmaceutical compositions according to the present invention, and for use in accordance with the present invention, may include, in addition to active ingredient, a pharmaceutically acceptable excipient, carrier, buffer, stabiliser or other materials well known to those skilled in the art. Such materials should be non-toxic and should not interfere with the efficacy of the active ingredient.

The precise nature of the carrier or other material will depend on the route of administration.

Various routes of administration are possible, such as via the respiratory or pulmonary tract, the gastro-intestinal tract or by cutaneous, subcutaneous, intranasal, or intra-dermal injection.

Administration of a mycobacterial preparation to the respiratory tract may occur using any suitable formulation, for example, in solution as an aerosol, bound covalently or non-covalently to a semi persistent particulate carrier administered, as a snuff for the upper respiratory tract or as an intra-tracheal injection. Particle size may be used to target appropriate parts of the airways.

Suitable formulations for administration via the gastro-intestinal tract include heat-killed organisms in capsules designed to release in the appropriate part of the gut. Alternatively, instead of whole organisms, selected components or extracts may be used. A suitable dose for administration by oral route may be 10⁷ to 10¹⁰ organisms or equivalent.

A pharmaceutical composition suitable for oral administration may be in tablet, capsule, powder or liquid form. A tablet may include a solid carrier such as gelatin or an adjuvant. Liquid pharmaceutical compositions generally include a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included.

Preparations for administration by injection, which may be cutaneous, subcutaneous, intra-nasal, intra-dermal or intra-tracheal, may comprise killed whole organisms or extracts, components or fractions thereof. For injection, the active ingredient will be in the form of a parenterally acceptable aqueous solution which is pyrogen-free and has suitable pH, isotonicity and stability. Suitable diluents, which are pharmaceutically acceptable and may be preferred, have been discussed already above.

A mycobacterial preparation may be adsorbed onto a suitable inert support (for example, nitrocellulose) before administration.

Whole killed M. vaccae cells or material derived from M. vaccae may be used including an extract or a synthetic molecule which has the requisite activity.

It is to be understood that in the context of the present invention, ‘treatment’ refers to therapy which is designed to alleviate the symptoms of a disease or condition, for example by modulation of the immune system, as well as to therapy designed to cure such diseases or conditions.

Experimental

Balb/C mice were given 10⁸ M. vaccae by the subcutaneous route.

M. vaccae were ultrasonically disrupted and adsorbed onto nitrocellulose particles.

Three weeks after the initial immunisation, nitrocellulose particles either with or without adsorbed M. vaccae were pulmonarily administered to the immunised mice by an intra-tracheal injection.

Mice were analysed 12 hours, 1, 3, 6, 10 and 17 days after intra-tracheal injection. Analysis was carried out as described below.

Mice were anaesthetised and then transcardially perfused with heparinised saline followed by 4% paraformaldehyde solution in 0.1 M phosphate buffer (approximately 7 ml per animal). Following perfusion, tissues were post-fixed in the same fixative for approximately six months.

The brain was then sectioned and examined for the activation of markers using immunohistochemical techniques. Tissues were dissected and transferred to 0.1 M phosphate buffer containing 30% sucrose and 0.1% sodium azide for at least 24 h. Intact brains were frozen on dry ice, then 30 μm sections were cut using a cryostat. Six sets of alternate sections from each brain were collected in a cryoprotectant solution (0.05 M sodium phosphate buffer containing 30% ethylene glycol and 20% glycerol) and stored at B20° C. until used for immunohistochemical staining. One set of sections was used for immunohistochemical double-labelling for c-Fos and tyrosine hydroxylase using the avidin-biotin-horseradish peroxidase method.

Labelling for c-Fos was performed by preincubating with freshly prepared 1% H₂O₂ in PBS for 10 min, followed by incubation with anti-c-Fos rabbit polyclonal antibody (c-Fos Ab-5, Oncogene Science, Cambridge, Mass.; distributed by Calbiochem-Novabiochem (UK) Ltd., Nottingham, UK) diluted 1:8,000 in PBS containing 0.3% Triton X-100 (PBST) 16-20 h. Sections were washed using PBST then incubated with biotinylated swine anti-rabbit antibody (DAKO, Cambridgeshire, UK; Cat. no. E0353) at a 1:200 dilution for 80 min.

Sections were washed again using PBST then incubated with ABC reagent at a dilution of 1:200 of each reagent, prepared 30 min before use (Vector Laboratories, Burlingame, Calif.; distributed by Vector Laboratories, Ltd., Peterborough, UK, Cat. no. PK-6101), for 80 min. After washing with PBST, then PBS, sections were incubated with substrate as recommended by the manufacturer (Vector SG peroxidase substrate, Cat. no. SK-4700) for 2-10 min.

Prior to immunohistochemical labelling of the same sections for tyrosine hydroxylase, sections were washed thoroughly with PBS. Sections were then incubated with anti-tyrosine hydroxylase rabbit polyclonal antibody (Chemicon, Temecula, Calif.; distributed by Chemicon International, Ltd., Harrow, UK; Cat. no. AB152) diluted 1:8000 in PBST for 16-20 h. Sections were washed using PBST then incubated with biotinylated swine anti-rabbit antibody at a 1:200 dilution for 80 min. Sections were washed again using PBST then incubated with ABC reagent at a dilution of 1:200 of each reagent (Vector Laboratories, Burlingame, Calif., Cat. no. PK-6101) for 80 min.

After washing with PBST, then PBS, sections were incubated with substrate (3,3=-diaminobenzidine tetrahydrochloride, and H₂O₂ Vector Laboratories, Cat. no. SK-4100, 1:2 dilution of recommended concentrations) in 0.1 M sodium phosphate buffer for 2-8 min.

After washing in PBS, sections were transferred to gelatin-coated glass slides and mounted with coverslips using DPX mounting medium (BDH Laboratory Supplies, Poole, England). Immunohistochemical double-labelling for c-Fos and tryptophan hydroxylase was conducted as described above, substituting a polyclonal sheep anti-tryptophan hydroxylase antibody (Biogenesis Ltd., Poole, UK, Cat. no. 9260-2505, 1:12,000 dilution) for the anti-tyrosine hydroxylase antibody, and subsequently, substituting a biotinylated rabbit anti-sheep antibody (Vector Laboratories, Cat. no. PK-6106, 1:200 dilution) for the biotinylated swine anti-rabbit antibody.

The activation of brain areas was determined from the expression patterns of c-Fos, tyrosine hydroxylase and tryptophan hydroxylase as described above.

The following nuclei were shown to be activated by intra-tracheal M. vaccae:

Nucleus Tractus Solitarius (nTS) (especially the dorsomedial and dorsolateral parts, known to be a primary targets of lung afferents), co-localisation with tyrosine hydroxylase (TH; a marker of noradrenergic neurones) was low.

Intermediate reticular nucleus (IRT)

Ventrolateral medulla (VLM) approx ⅓ co-localised with TH

Locus coeruleus (LC) (almost 100% noradrenergic) and subcoeruleus

Arcuate nucleus (Arc)

Dorsomedial hypothalamic nucleus (DM)

Anterior hypothalamus (AH)

Bed nucleus of the stria terminalis (BST)

Kölliker-Fuse nucleus

Anterior pretectal nucleus

Dorsal raphe (DR)

Raphe magnus nucleus (RMg) and adjacent reticular formation; most c-Fos co-localised with tryptophan hydroxylase (the rate limiting enzyme for serotonin synthesis.

Caudal linear raphe nucleus (CLi)

Lateral and ventral Periaqueductal gray (PAG)

Central amygdaloid (Ce)

Medial amygdaloid (Me)

Lateral hypothalamus (LH)

Paraventricular nucleus of the hypothalamus (PVN), corticotropin releasing factor (CRF) neurones.

Lateral septal nucleus (LS)

Organum vasculosum of the lamina terminalis (OVLT)

Activation of the Hypothalamo-Pituitary-Adrenal Axis.

Recipients of M. vaccae showed increased c-Fos expression in the ventrolateral and dorsolateral parts of Nucleus Tractus Solitarius (nTS).

This observation is consistent with previous studies demonstrating these specific sub-nuclei as the primary targets of sensory fibres from the extra-thoracic and intra-thoracic trachea, the right main bronchus and the upper right lobe of the lung (Kalia and Mesulam, 1980). It is also consistent with the view that the nTS is the ‘common gateway’ through which signals of inflammation are relayed to the HPA axis (Ericsson et al. 1994).

These animals showed a profound increase in the production of the stress hormone, corticosterone from the adrenal.

Activation of Novel Pathways

Pain Inhibitory Pathways

Previous studies have identified descending pathways that inhibit pain (reviewed by Willis and Westlund, 1997). M. vaccae resulted in marked activation of neurones within this ‘descending analgesia’ system (see Willis, 1982, Besson and Chaouch, 1987, Fields and Besson, 1988 and Light, 1992).

Areas activated after M. vaccae treatment that are part of the ^{˜}descending analgesia^{˜} system include:

i) Lateral and ventral Periaqueductal gray (PAG)

ii) Raphe magnus nucleus (RMg) and adjacent reticular formation

iii) Locus coeruleus and subcoeruleus

iv) Kölliker-Fuse nucleus

v) Ventrolateral medulla

vi) Anterior pretectal nucleus

Activation of neurones in the anterior pretectal nucleus is of particular interest because stimulation in this structure results in long-lasting antinociception (i.e. reduced pain perception) without aversive side effects (Rees and Roberts, 1986, 1993; Prado, 1989; Prado and Roberts, 1985).

In M. vaccae-treated mice, there was a robust activation of neurones throughout the subnucleus reticularis dorsalis. This structure is thought to be the basis of ^{˜}diffuse noxious inhibitory contols (DNIC)^{˜} (Villanueva and Le Bars, 1995), which is another pain inhibitory system.

The simultaneous activation of neurones within both the ^{˜}descending analgesia^{˜} system and the ^{˜}diffuse noxious inhibitory contols^{˜} (DNIC) shows that M. vaccae has potent analgesic effects.

Affective Disorder

Intra-tracheal M. vaccae results in activation of noradrenergic and serotonergic systems that are implicated in stress and major depression.

In M. vaccae-treated mice, there was activation of noradrenergic neurones within the locus coeruleus. Clinical studies show that a substantial portion of severely depressed patients show a reduction of noradrenaline turnover within the brain, and this is likely to be due primarily to a reduction in the activity of the noradrenergic neurones within the locus coeruleus.

M. vaccae may therefore be used to treat severely depressed patients to restore locus coeruleus noradrenergic function and consequently the vigilance and arousal levels that are thought to be dependent on this nucleus.

Treatment with M. vaccae also resulted in the activation of serotonergic neurones within topographically defined sub-regions of the serotonergic raphe nuclei. As is the case with noradrenergic systems, depressed patients show a reduction of serotonin turnover within the brain (reviewed by Willner 1985). Importantly, the activation of serotonergic neurones by treatment with M. vaccae was highly topographically organised. The small sub-population of serotonergic neurones activated (within the interfascicular dorsal raphe nucleus) corresponds to the location of a small sub-population of serotonergic neurones (Type II serotonergic neurones) with unique electrophysiological and behavioural correlates (Rassmussen et al. 1984).

The location of this small sub-population of serotonergic neurones also corresponds to the location of bilateral serotonergic neurons known to project to the dorsolateral prefrontal cortex (Porrino and Goldman-Rakic, 1982). This region of the prefrontal cortex in turn is recognised as a focal region for hypometabolism associated with depressive symptoms in depressed patients (Drevets, 1998).

In depressed patients, Type II serotonergic neurones in the interfascicular dorsal raphe nucleus may have reduced activity; selective activation of this small subpopulation of serotonergic neurones by M. vaccae may be used in the treatment of depression and stress-related psychiatric disorders, by restoring serotonergic function.

Other Stress Related Disorders

M. vaccae induced c-Fos in the dorsomedial hypothalamic nucleus, a brain region thought to play an important role in major depression and the neuroendocrine correlates of depression (Lowry and Lightman, in preparation) as well as anxiety (File et al 1999) and panic disorder (Shekhar and Keim 1997). This nucleus also plays a key role in regulation of feeding (Bernardis and Bellinger 1998). Based on the clear association of the dorsomedial hypothalamic nucleus with autonomic, behavioural and neuroendocrine responses to stress (Thompson and Swanson, 1998), it is likely that this nucleus plays a significant role in the regulation of feeding patterns during periods of stress, and potentially behavioural patterns in anxiety- or stress-related eating disorders including bulimia and anorexia nervosa.

M. vaccae therefore activates an array of stress related brain pathways including noradrenergic and serotonergic pathways previously associated with stress-related pathology. M. vaccae and appropriate derivatives may be used in regulating anxiety, panic and eating disorders.

Schizophrenia

The dorsomedial hypothalamic nucleus is strongly implicated in Schizophrenia because it contains neurones that project to the distributed heteromodal cortex, which is itself abnormal in these patients. Subnormal function of dopaminergic and serotonergic neurones within the dorsomedial hypothalamic nucleus may underlie deficits of dopamine and serotonin known to exist in the heteromodal cortex of schizophrenia patients (Horst and Luiten, 1986; Ross and Pearlson, 1996).

Although the dorsomedial hypothalamic nucleus contains populations of dopaminergic and serotonergic neurones (Refs, see Lowry et al 1996), under normal conditions it has not been possible to visualise these populations of neurones in mammals using immunohistochemical procedures directed against dopamine, serotonin, or their synthetic enzymes tyrosine hydroxylase and tryoptophan hydroxylase, respectively.

In M. vaccae-treated mice, however, we have noted large numbers of tyrosine hydroxylase-immunoreactive (dopaminergic) neurones within the dorsomedial hypothalamic nucleus. The dorsomedial hypothalamic nucleus is one of a select few brain areas that expresses IL-1β, Type 2 interleukin 1 receptor, IL-6 and IL-6 receptor mRNA (Shobitz et al 1992, 1993; Yabuuchi et al 1993, Nishiyori et al 1997). This provides indication that the DMN may be a preferential site of central cytokines following peripheral inflammation. The presence of strong tyrosine hydroxylase immunoreactivity in the dorsomedial hypothalamic nucleus indicates an up-regulation of dopamine synthesis in these novel neurones.

By enhancing dopamine or serotonin synthesis within the dorsomedial hypothalamic nucleus, Mycobacteria such as M. vaccae may be used in treatment for Schizophrenia.

References

Bernadis L. and Bellinger L. (1998) Proc Soc Exp Biol Med (1998); 218: 284-306.

Besson J. and Chaouch A. (1987) Physiol Rev 67 67-186

Bonicke et al., (1964) Zentr albl. Bakteriol. Parasitenkd. Infection skr. Hyg. Abt. 1, Orig., 192: 133

Boyden et al., (1955). J. Immunol. 75: 15.

Drevets W. (1998) Annu Rev Med 49 341-361

Ericsson et al. (1994) J. Neuroscience 14 897-913

File S. et al.(1999) Neuropsychopharmocology 21 312-320

Fields H. and Besson J. (1988) Progress in Brain Research No 77. Amsterdam: Elsevier

Gray J. (1982) The Neuropsychology of Anxiety: An Enquiry into the Functions of the Septo-Hippocampal System. Oxford: Clarendon Press.

Kalia M. and Meulam C M. J. Comp. Neurol. (1980) 193 467-508

Light A. (1992) The Initial Processing of Pain and its Descending Control: Spinal and Trigeminal Systems. Basel: Karger.

Lowry C. et al. (1996) Brain Behav Evol (1996); 48: 70-93.

Lowry C. and Lightman S. The Lancet (manuscript in preparation)

Nishiyori A. et al. (1997)Brain Res Mol Brain Res 50 237-245

Prado W. (1989) Brain Res 493 147-154

Prado W. and Roberts M. (1985) Brain Res 340 219-228

Purrino L. and Goldman-Rakic P. (1982) J. Comp. Neurol. 205 63-75.

Rasmussen K. et al. (1984) Expt. Neurol. 83 302-317

Rees H. and Roberts M.(1986) Pain 25 83-93

Rees H. and Roberts M.(1993) Pain 53 121-135

Ross C. and Pearlson G. (1996)TINS 19 171-176

Shekar A. and Keim S. (1997) J. Neurosci (1997) 17 9726-9735

Shobitz et al. (1992) Neurosci Lett 136 189-192

Shobitz et al. (1993)Eur J Neurosci 5 1426-1435

Stanford and Paul, (1973) Ann. Soc. Belge Med. Trop. 53: 141-389.

Telenti et al. (1993) J. Clin. Microbiol. 31: 175-178.

Ter Horst G. and Luiten P. (1986) Brain Res Bull 16 231-248

Thompson R. and Swanson L. (1998) Brain Res Rev 27 89-118

Villanueva L. and Le Bars D.(1995) Biol Res 28 113-125

Willis W. and Westlund K. (1997) J. Clin Neurophysiol 14 2-31

Willis W. (1982) Control of Nociceptive transmission in the Spinal Cord. In Ottoson D., ed., Progress in Sensory Physiology 3 Berlin: Springer-Verlag.

Willner P. (1985) Depression: a psychobiological synthesis. John Wiley & Sons: New York

Yabuuchi et al.(1993)Brain Res Mol Brain Res 20: 153-161

1. Use of mycobacterial material in the manufacture of a medicament for use in a method of treating a condition of the central nervous system (CNS) with the proviso that the condition is not a mental disease associated with an auto-immune reaction.

2. Use according to claim 1 wherein the condition is selected from the group consisting of pain, a mental disorder and a stress-related disorder.

3. Use according to claim 2 wherein the mental disorder is schizophrenia not associated with an auto-immune reaction.

4. Use according to claim 2 wherein the stress-related disorder is depression.

5. Use according to any one of claims 1 to 4 wherein the mycobacterial material comprises whole, killed mycobacterial cells.

6. Use according to claim 5 wherein the mycobacterial cells are ultrasonically disrupted.

7. Use according to any one of claims 1 to 6 wherein the mycobacterial material is M. vaccae material.

8. Use according to any one of claims 1 to 7 wherein the mycobacterial material is adsorbed onto an inert support, e.g. nitrocellulose.

9. Use according to any one of claims 1 to 8 wherein the medicament is for administration of mycobacterial material to the respiratory tract.

10. Use according to claim 9 wherein the medicament is for administration of mycobacterial material to the respiratory tract by intra-tracheal injection.

11. A method of treatment of a condition of the central nervous system (CNS), with the proviso that the condition is not a mental disease associated with an auto-immune reaction, the method comprising administering to a patient an effective amount of a therapeutic composition comprising mycobacterial material.

12. A method according to claim 11 wherein the condition is selected from the group consisting of pain, a mental disorder and a stress-related disorder.

13. A method according to claim 12 wherein the mental disorder is schizophrenia not associated with an auto-immune reaction.

14. A method according to claim 12 wherein the stress-related disorder is depression.

15. A method according to any one of claims 11 to 14 wherein the mycobacterial material comprises whole, killed mycobacterial cells.

16. A method according to claim 15 wherein the mycobacterial cells are ultrasonically disrupted.

17. A method according to any one of claims 11 to 16 wherein the mycobacterial material is M. vaccae material.

18. A method according to any one of claims 11 to 17 wherein the mycobacterial material is adsorbed onto an inert support, e.g. nitrocellulose.

19. A method according to any one of claims 11 to 18 wherein the medicament is for administration of mycobacterial material to the respiratory tract.

20. A method according to claim 19 wherein the medicament is for administration of mycobacterial material to the respiratory tract by intra-tracheal injection.
